SEQUENCE LISTING (2 sequences)

SEQ ID NO: 1 - DNA, 24 bp, Artificial Sequence
  Designed oligonucleotide based on beta-actin gene
tagaagcatttgcggtggacgatg

SEQ ID NO: 2 - DNA, 24 bp, Artificial Sequence
  Designed oligonucleotide based on beta-actin gene
catcgtccaccgcaaatgcttcta

(19) United States
(12) Patent Application Publication
(10) Pub. No.: US 2010/0062475 A1
(43) Pub. Date: Mar. 11, 2010

(54) PARTICLE FOR MEDICAL USE, PARTICLE FOR ANLAYSIS AND METHOD OF PRODUCING THE SAME

(75) Inventors: Yuzo Hamaguchi, Tokyo (JP); Sohei Funaoka, Tokyo (JP); Yoshiaki Fukunishi, Tokyo (JP); Tayuki Matsumoto, Tokyo (JP)

Correspondence Address: SMITH, GAMBRELL & RUSSELL, 1130 CONNECTICUT AVENUE, N.W., SUITE 1130, WASHINGTON, DC, 20036, US

(73) Assignee: SUMITOMO BAKELITE COMPANY, Tokyo (JP)

(21) Appl. No.: 12/448,681

(22) PCT Filed: Jan. 11, 2008
(86) PCT No.: PCT/JP2008/050284

§ 371 Date: Jun. 30, 2009

(30) Foreign Application Priority Data
Jan. 16, 2007 (JP) 2007-007411
Aug. 10, 2007 (JP) 2007-208783
Aug. 29, 2007 (JP) 2007-222094

(51) Int. Cl. C12Q 1/02; B32B 5/16; B05D 7/00

(52) U.S. Cl. 435/29; 428/407; 428/406; 428/405; 427/212

(57) ABSTRACT

The object of the present invention is to provide a particle for medical use which has an excellent capability of fixing a biologically active substance and such a chemical/physical stability that the particle is less dissolved or deteriorated in a washing step, and a particle for analysis which has an excellent capability of capturing a biomolecule and such a chemical/physical stability that the particle is less dissolved or deteriorated in a washing step.

The object of the present invention is achieved by a particle for medical use which has on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop polymerization reaction The object of the present invention is also achieved by a particle for analysis of the interaction of biomolecules, which particle having on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop, polymerization reaction, wherein the biologically active substance is fixed through the functional group for fixing a biologically active substance in the polymer-containing layer.

### TECHNICAL FIELD

The present invention relates to a particle for medical use having a function of fixing a biologically active substance, a particle for analysis for the analysis of the interaction of biomolecules, and a method of producing the same.

### BACKGROUND ART

Polymer-coated particles composed of various kinds of particles and polymers have been widely used in the industrial field. In recent years, such polymer particles are increasingly important in the fields of medicine and fundamental biology. For example, there is growing interest in applications to affinity chromatography carriers, medical diagnosis, drug delivery system (DDS), and drug development. As an example of the application to drug development, biomolecules such as specific proteins are captured by biologically active substances called ligands which have been fixed to various particles (carriers), and then the biomolecules are separated and refined.

Particles used as carriers are required to satisfy the following conditions: (1) the particles have a functional group capable of fixing a large amount of ligand or spacer under a mild condition; (2) the carrier does not nonspecifically absorb biological substances such as proteins; (3) the particles have a mechanical strength suitable for the intended use. However, no carrier which satisfies these conditions has been developed yet.

The performance of a carrier largely depends on the amount of ligand to be fixed, so that a carrier is required to fix as much ligand as possible. The larger the amount of ligand is fixed, the larger the total amount of protein is captured. In particular, when the target protein is a minor component among the proteins to be captured by the ligand, the target protein may be not detected if the fixed amount is too small. Accordingly, particles capable of fixing a large amount of ligand have been demanded.

It is also very important to inhibit nonspecific adsorption of components to a carrier, which components excluding the protein to be captured. Among known carriers, inorganic carriers composed of silica gel particles are considered to be suitable for separation because they are porous and have high physical strength. However, silica gel particles are disadvantageous in that they are highly prone to nonspecific adsorption, so that they are scarcely used for practical purposes. As synthetic polymer carriers, particles composed of a polyacrylamide gel (trade name: Bio-GelP, manufactured by Bio-Rad Laboratories, Inc.), polystyrene, or an ethylene-maleic anhydride copolymer have been developed. However, these polymers also have the disadvantage of being prone to nonspecific adsorption of biological substances.

As described above, when particles are used as medical carriers or carriers for analysis, nonspecific adsorption often becomes a problem. In order to avoid the problems, various techniques have been studied. An example thereof is a blocking method wherein a target biologically active substance is attached to the particle surface, and then a harmless protein such as bovine serum albumin (BSA) is attached to the rest portion of the particle surface. However, the effect of the method is not sufficient. In another method, DNA which specifically binds to a specific protein is attached to particles having a surface containing epoxy groups, and the particles are used for refinement of the protein (for example, Patent Document 1). In this method, epoxy groups are introduced to the particle surface with glycidyl methacrylate or the like because it exhibits low nonspecific adsorption to proteins. However, direct binding between epoxy groups and a biologically active substance such as DNA requires significantly severe reaction conditions such as reaction under alkaline conditions or conditions of high temperature. Therefore, the method is improper when the biologically active substance to be fixed is unstable in alkali and at high temperatures, because the biologically active substance may be deteriorated during the fixing process.

Another method of reducing nonspecific adsorption to particles is the synthesis of polymer particles with low nonspecific adsorption through emulsion polymerization or the like. For example, Patent Document 2 describes a method for producing particles through emulsion polymerization or suspension polymerization of an ethylenically unsaturated polymerizable monomer having a reactive group capable of reacting with a biologically active substance, an ethylenically unsaturated polymerizable monomer having a polyoxyalkylene side chain, and an ethylenically unsaturated polymerizable vinyl aromatic monomer capable of imparting hydrophobicity. However, with these methods, it is difficult to control the size of the resultant particles. In general, particles having a relatively uniform particle size are produced by emulsion polymerization, but the particle size is limited to the order of submicron. On the other hand, particles produced by suspension polymerization have a large particle size distribution, so that they must undergo classification when used as, for example, a column filler. However, it is difficult to minutely classify the polymer particles without a special apparatus. In addition, the size of the obtained particles range from several tens microns to several hundred microns, and it is difficult to synthesize particles having a smaller diameter. Furthermore, particles obtained by polymerization are polymers, so that the strength of the particles as a carrier is inevitably limited. For applications where high particle strength is required, it is inevitably necessary to increase particle strength by, for example, increasing the ratio of the ethylenically unsaturated polymerizable vinyl aromatic monomer in the polymer. However, this is disadvantages in terms of nonspecific adsorption.

[Patent Document 1] Japanese Patent No. 2753762

[Patent Document 2] Japanese Patent No. 3215455

### DISCLOSURE OF INVENTION

### Problems to be Solved by the Invention

A method was expected to be effective in reducing nonspecific adsorption with maintaining particle strength, which is a method of coating, for example, silica gel particles having high physical strength with a polymer having low nonspecific adsorption. More specifically, the method of forming covalent bonds between a polymer and a surface of a particle was expected to be particularly effective in preventing the polymer from being dissolved in a washing step. However, unlike in the case of applying a polymer onto a relatively large flat substrate, a conventional method of causing a functional group in a polymer to react with a functional group on a surface of a particle provides poor reactivity because the steric hindrance of the polymer limits the frequency of collisions between the functional groups, so that it is difficult to uniformly coat the polymer onto the particle surface. Therefore, the amount of the polymer coated on the particle surface in a chemically/physically stable manner gets insufficient, thereby providing a poor capability of fixing a target biologically active substance to the particle.

A first object of the present invention is to provide a particle for medical use which has an excellent capability of fixing a target biologically active substance and has such a chemical/physical stability that the particle is less dissolved or deteriorated in a washing step, and is also to provide a particle for medical use which exhibits lower nonspecific adsorption of proteins or the like and provides a high S/N ratio, in addition to having the above characteristics.

A second object of the present invention is to provide a particle for analysis which has an excellent capability of capturing a target biomolecule and has such a chemical/physical stability that the particle is less dissolved or deteriorated in a washing step, and is also to provide a particle for analysis which exhibits lower nonspecific adsorption of proteins or the like and provides a high S/N ratio, in addition to having the above-mentioned characteristics.

### Means for Solving the Problems

The present invention is:

(1) A particle for medical use which has on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop polymerization reaction;

(2) The particle for medical use according to (1), wherein the polymerizable component contains an ethylenically unsaturated polymerizable monomer having an alkylene glycol residue;

(3) The particle for medical use according to (1) or (2), wherein the functional group of the polymerizable monomer having a functional group for fixing a biologically active substance is at least one functional group selected from an aldehyde group, an active ester group, an epoxy group, a vinylsulfone group and biotin;

(4) The particle for medical use according to any of (1) to (3), wherein the polymerizable monomer having a functional group for fixing a biologically active substance is a monomer having an active ester group and represented by the following formula [1]:

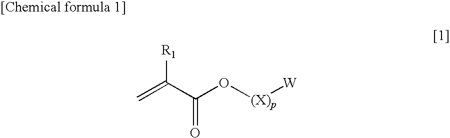

wherein R₁ represents a hydrogen atom or a methyl group; X represents an alkylene group or an alkylene glycol residue having 1 to 10 carbon atoms; W represents an active ester group; p represents an integer from 1 to 100; and provided that when p is an integer of 2 or more and 100 or less, the repeated Xs may be the same or different from each other;

(5) The particle for medical use according to (4), wherein the active ester group is a p-nitrophenyl ester or N-hydroxysuccinimide ester;

(6) The particle for medical use according to any of (2) to (5), wherein the ethylenically unsaturated polymerizable monomer having an alkylene glycol residue contains a monomer represented by the following formula [2]:

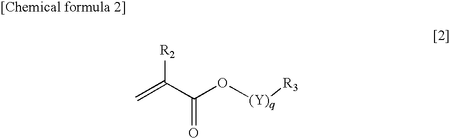

wherein R₂ represents a hydrogen atom or a methyl group; R₃ represents a hydrogen atom or an alkyl group having 1 to 20 carbon atoms; Y represents an alkylene glycol residue having 1 to 10 carbon atoms; q represents an integer from 1 to 100; and provided that when q is an integer of 2 or more and 100 or less, the repeated Ys may be the same or different from each other;

(7) The particle for medical use according to any of (2) to (6), wherein the ethylenically unsaturated polymerizable monomer having an alkylene glycol residue contains methoxy polyethylene glycol (meth)acrylate and/or ethoxy polyethylene glycol (meth)acrylate;

(8) The particle for medical use according to (7), wherein an average repeating number of the ethylene glycol residue of the methoxy polyethylene glycol (meth)acrylate and/or ethoxy polyethylene glycol (meth)acrylate is from 3 to 100;

(9) The particle for medical use according to any of (1) to (8), wherein the polymerizable functional group is one or more kinds selected from the group consisting of a methacrylic group, an acrylic group and a vinyl group;

(10) The particle for medical use according to any of (1) to (8), wherein the chain-transfer group is a mercapto group;

(11) The particle for medical use according to any of (1) to (10), wherein the core particle comprises an inorganic material;

(12) The particle for medical use according to (11), wherein the inorganic material comprises an inorganic oxide;

(13) The particle for medical use according to (12), wherein the inorganic oxide is a silicon oxide;

(14) The particle for medical use according to any of (1) to (13), wherein the polymerizable functional group or the chain-transfer group is introduced to the surface of the core particle by forming a covalent bond between a silane coupling agent containing the polymerizable functional group or the chain-transfer group and a functional group on the surface of the core particle;

(15) The particle for medical use according to (14), wherein the silane coupling agent containing the polymerizable functional group or the chain-transfer group is an alkoxysilane containing the polymerizable functional group or the chain-transfer group;

(16) A method of producing the particle for medical use defined by any of (1) to (15), which method comprising the steps of hydrolyzing an alkoxysilane containing the polymerizable functional group or the chain-transfer group in an acid aqueous solution, heating the core particle while stirring the same in the acid aqueous solution of the alkoxysilane containing the polymerizable functional group or the chain-transfer group, and further heating the particle after drying the same;

(17) The method of producing the particle for medical use according to (16), which method further comprising the steps of mixing in a solvent the core particle to which the polymerizable functional group or the chain-transfer group is introduced with a polymerizable monomer to develop polymerization reaction, and drying the particle;

(18) The method of producing the particle for medical use according to (17), wherein the polymerization reaction is a radical polymerization reaction;

(19) A particle for analysis of the interaction of biomolecules, which particle having on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop polymerization reaction, wherein the biologically active substance is fixed through the functional group for fixing a biologically active substance in the polymer-containing layer;

(20) The particle for analysis according to (19), wherein the polymerizable component contains an ethylenically unsaturated polymerizable monomer having an alkylene glycol residue;

(21) The particle for analysis according to (19) or (20), wherein the functional group for fixing a biologically active substance is at least one functional group selected from the group consisting of an aldehyde group, an active ester group, an epoxy group, a vinylsulfone group and biotin;

(22) The particle for analysis according to any of (19) to (21), wherein the biologically active substance is at least one selected from the group consisting of a nucleic acid, an aptamer, a protein, an antibody, an antigen, protein A, protein G, a ligand, a peptide, glutathione, a low-molecular compound, biotin, a sugar chain, a lectine and a glycoprotein;

(23) A method of producing the particle for analysis defined by any of (19) to (22), which method comprising the step of bringing the particle having a polymer-containing layer formed on the surface thereof into contact with a solution prepared by dissolving a biologically active substance in a phosphate buffer;

(24) The method of producing the particle for analysis according to (23), wherein the concentration of the phosphate in the phosphate buffer is 0.1 M or more and 5 M or less;

(25) The method of producing the particle for analysis according to (23) or (24), wherein the phosphate is potassium dihydrogen phosphate, sodium dihydrogen phosphate, dipotassium hydrogen phosphate or disodium hydrogen phosphate; and

(26) A method of using the particle for analysis defined by any of (19) to (22), which method comprising collecting a target biological substance is collected by bringing the particle for analysis defined by any of (19) to (22) into contact with at least one solution selected from a solution in which a target biomolecule is dissolved, blood, blood plasma, blood serum, a cell lysate, a cell culture solution and a tissue lysate.

### EFFECTS OF THE INVENTION

According to the present invention, it is possible to provide a particle for medical use which has an excellent capability of fixing a target biologically active substance and has such a chemical/physical stability that a polymer-containing layer on the surface of the particle is less dissolved or deteriorated in a washing step. Furthermore, by adding a component containing an ethylenically unsaturated polymerizable monomer having an alkylene glycol residue to the component of the polymer, it is possible to provide a particle for medical use which exhibits lower nonspecific adsorption of proteins or the like. Moreover, according to the present invention, it is possible to provide a particle for analysis which has an excellent capability of capturing a target biomolecule and has such a chemical/physical stability that a polymer-containing layer on the surface of the particle is less dissolved or deteriorated in a washing step. Furthermore, by adding a component containing an ethylenically unsaturated polymerizable monomer having an alkylene glycol residue to the component of the polymer, it is possible to provide a particle for analysis which exhibits lower nonspecific adsorption of proteins or the like.

### BEST MODE FOR CARRYING OUT THE INVENTION

The particle for medical use of the present invention is a particle for medical use which has on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop polymerization reaction.

The particle for analysis of the present invention is a particle for analysis of the interaction of biomolecules, which particle having on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop polymerization reaction, wherein the biologically active substance is fixed through the functional group for fixing a biologically active substance in the polymer-containing layer.

The particle of the present invention has on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop polymerization reaction. The polymer formed on the surface of the core particle contains a functional group for fixing a biologically active substance; thus, it has the property of fixing a specific biologically active substance. Furthermore, the particle of the present invention can cause polymerizable functional groups or chain-transfer groups forming covalent bonds with the surface of a core particle to form a polymer, thereby making it possible to cause the polymer to be densely grafted onto the surface of the core particle. About the thus-obtained grafted particle, the polymer does not flow out therefrom in a washing step; moreover, the polymer can be coated onto the surface thereof more uniformly than by the conventional method of reacting functional groups in a polymer with a functional group on the surface of a particle.

Said polymerizable component contains at least a polymerizable monomer having a functional group for fixing a biologically active substance; moreover, said polymerizable component also preferably contains an ethylenically unsaturated polymerizable monomer having an alkylene glycol residue. Alkylene glycol residues have the property of inhibiting nonspecific adsorption of proteins or the like. The polymerizable monomer having a functional group for fixing a biologically active substance may also functions as an ethylenically unsaturated polymerizable monomer having an alkylene glycol residue, or an ethylenically unsaturated polymerizable monomer having an alkylene glycol residue may be contained in the polymerizable component in addition to the polymerizable monomer having a functional group for fixing a biologically active substance.

In the polymerizable monomer having a functional group for fixing a biologically active substance to be used in the invention, the functional group may be a chemically active group, receptor group or ligand group, but is not limited thereto. Specific examples thereof include an aldehyde group, an active ester group, an epoxy group, a vinylsulfone group, biotin, a thiol group, an amino group, an isocyanate group, an isothiocyanate group, a hydroxyl group, an acrylate group, a maleimide group, a hydrazide group, an azide group, an amide group, a sulfonate group, streptavidin and metal chelates. Among them, preferred are an aldehyde group, an active ester group, an epoxy group and a vinylsulfone group from the viewpoint of the reactivity thereof with an amino group which is contained in a biologically active substance in many cases. Moreover, biotin is preferred since it has a high binding constant onto a biologically active substance. In particular, an active ester group is most preferred from the viewpoint of the storage stability of the monomer.

The polymerizable monomer having a functional group for fixing a biologically active substance to be used in the invention is not particularly limited about the structure thereof. The monomer is preferably a compound represented by the following formula [1], wherein a (meth) acrylic group and an active ester group are bonded to each other through a chain of an alkylene glycol residue having 1 to 10 carbon atoms or an alkylene group. When the compound represented by the following formula (1) has a chain of an alkylene glycol residue, the compound itself has the property of inhibiting nonspecific adsorption of proteins. For this reason, a monomer wherein a (meth)acrylic group and an active ester group are bonded to each other through a chain of an alkylene glycol residue has both of the properties of fixing a biologically active substance and inhibiting nonspecific adsorption of proteins. Accordingly, even if a polymer from such a monomer is a homopolymer, the polymer can be preferably used as a polymer for forming a layer on the surface of a particle. In the present invention, (meth)acrylic means acrylic and/or methacrylic, and (meth)acrylate means acrylate and/or methacrylate.

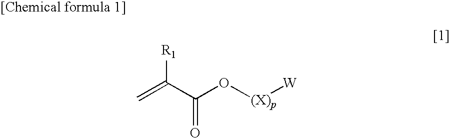

wherein R₁ represents a hydrogen atom or a methyl group; X represents an alkylene group or an alkylene glycol residue having 1 to 10 carbon atoms; W represents an active ester group; p represents an integer from 1 to 100; and provided that when p is an integer of 2 or more and 100 or less, the repeated Xs may be the same or different from each other.

An alkylene group or alkylene glycol residue X in the formula [1] has 1 to 10 carbon atoms, preferably 1 to 6 carbon atoms, more preferably 2 to 4 carbon atoms, even more preferably 2 to 3 carbon atoms, most preferably 2 carbon atoms. The alkylene glycol residue referred to herein means an alkyleneoxy group (—R—O, wherein R is an alkylene group) which remains after a hydroxyl group at a single terminal or hydroxyl groups at both terminals of an alkylene glycol (HO—R—OH, wherein R is the alkylene group) are subjected to condensation reaction with a different compound. For example, in the case of methylene glycol (OH—CH₂—OH), the alkylene glycol residue is a methyleneoxy group (—CH₂—O—), and in the case of ethylene glycol (OH—CH₂CH₂—OH), the alkylene glycol residue is an ethyleneoxy group (—CH₂CH₂—O—).

The repeating number ‘p’ of Xs is an integer from 1 to 100. The number ‘p’ is more preferably an integer from 2 to 90, and most preferably an integer from 2 to 80. In the case of using a mixture of the compounds having varying numbers of ‘p’s, the number ‘p’ of the polymer is specified as the average value of the above. When the repeating number ‘p’ is 2 or more, the repeated Xs may be the same or different from each other.

The “active ester group” used in the invention means an ester group activated relative to a nucleophilic reaction by having a high acidic electron attracting group as one substituent of the ester group, that is an ester group having a high reaction activity, which is conventionally used in various chemical synthesis such as in a field of polymer chemistry, or in a field of peptide synthesis. Actually, phenol esters, thiophenol esters, N-hydroxyamine esters, esters of a heterocyclic hydroxy compound and so on are each known as an active ester group having a much higher activity than that of alkyl esters or the like.

Such an active ester group may be an ester wherein R″ in —COOR″ has the above-mentioned high acidic electron attracting group. Examples thereof include a p-nitrophenyl active ester group wherein R″ is p-nitrophenyl; an N-hydroxysuccinimide active ester group wherein R″ is N-hydroxysuccinimide; a phthalic imide active ester group wherein R″ is phthalic imide; and a 5-norbornene-2,3-dicarboxylmide active ester group wherein R″ is 5-norbornene-2,3-dicarboxylmide. In particular, a p-nitrophenyl active ester group or N-hydroxysuccinimide active ester group is preferred from the viewpoint of the balance between storage stability and high reactivity. A p-nitrophenyl active ester group is most preferred.

Examples of the polymerizable monomer having a functional group for fixing a biologically active substance include p-nitrophenyloxycarbonyl-poly(ethylene glycol)(meth)acrylate and succinimideoxycarbonyl-poly(ethylene glycol)(meth)acrylate. In particular, p-nitrophenyloxycarbonyl-poly(ethylene glycol)(meth)acrylate represented by the following formula is preferred. The repeating number p of the ethylene glycols and/or the average value of p is preferably from 2 to 20.

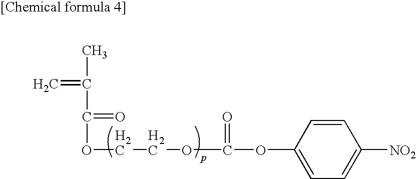

In a polymer, the ratio of the polymerizable monomer having a functional group for fixing a biologically active substance used in the present invention is not particularly limited, and is preferably from 1 to 99.7% by mole of the total number of repeating units of all monomers in the polymer, more preferably form 1 to 80% by mole thereof, most preferably from 1 to 70% by mole thereof.

Preferably, the polymer component used in the present invention also contains an ethylenically unsaturated polymerizable monomer having an alkylene glycol residue in addition to the polymerizable monomer having a functional group for fixing a biologically active substance. When the polymerizable monomer having a functional group for fixing a biologically active substance has an alkylene glycol residue, normally, the alkylene glycol residue also has a function of controlling the position of the functional group for fixing a biologically active substance. Because of this reason, from the viewpoint of improving the balance between the properties of inhibiting nonspecific adsorption of proteins and fixing a biologically active substance, it is preferred to form a copolymer with the polymerizable component which contains an ethylenically unsaturated polymerizable monomer having an alkylene glycol residue in addition to the polymerizable monomer having a functional group for fixing a biologically active substance.

The ethylenically unsaturated polymerizable monomer having an alkylene glycol residue, which monomer being different from the polymerizable monomer having a functional group for fixing a biologically active substance, is not particularly limited about the structure thereof, and is preferably a compound represented by the following formula [2], which is composed of a (meth)acrylic group and a chain of an alkylene glycol residue Y having 1 to 10 carbon atoms.

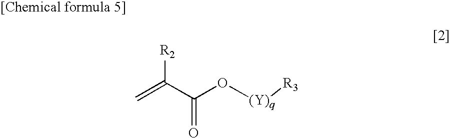

wherein R₂ represents a hydrogen atom or a methyl group; R₃ represents a hydrogen atom or an alkyl group having 1 to 20 carbon atoms; Y represents an alkylene glycol residue having 1 to 10 carbon atoms; q represents an integer from 1 to 100; and provided that when q is an integer of 2 or more and 100 or less, the repeated Ys may be the same or different from each other.

The alkylene glycol residue Y in the formula [2] has 1 to 10 carbon atoms, preferably 1 to 6 carbon atoms, more preferably 2 to 4 carbon atoms, even more preferably 2 to 3 carbon atoms, most preferably 2 carbon atoms. The repeating number ‘q’ of the alkylene glycol residues Ys is an integer from 1 to 100, more preferably an integer from 2 to 100, even more preferably an integer from 2 to 95, most preferably an integer from 20 to 90. In the case of a mixture of the compounds having varying numbers of ‘q’s, the number ‘q’ of the polymer is specified as the average value of the above.

Examples of the ethylenically unsaturated polymerizable monomer having an alkylene glycol residue include methoxy polyethylene glycol (meth)acrylate, ethoxy polyethylene glycol (meth)acrylate, 2-hydroxyethyl(meth)acrylate, 2-hydroxypropyl (meth)acrylate and an ester wherein the hydroxyl group thereof is mono-substituted, 2-hydroxybutyl (meth)acrylate and an ester wherein the hydroxyl group thereof is mono-substituted, glycerol mono (meth)acrylate, (meth)acrylate having polypropylene glycol as its side chain, 2-methoxyethyl (meth)acrylate, 2-ethoxyethyl(meth)acrylate, methoxydiethylene glycol (meth)acrylate, ethoxydiethylene glycol (meth)acrylate and ethoxy polyethylene glycol (meth)acrylate. Preferred is methoxy polyethylene glycol (meth)acrylate or ethoxy polyethylene glycol (meth)acrylate because of their low nonspecific adsorption of components other than a target biologically active substance, and their availability. In particular, methoxy polyethylene glycol (meth)acrylate or ethoxy polyethylene glycol (meth)acrylate wherein the average repeating number of ethylene glycol residues is from 3 to 100 is preferably used since the (meth)acrylate is good in handleability when synthesized.

In the case of using the ethylenically unsaturated polymerizable monomer having an alkylene glycol residue, which monomer being different from the polymerizable monomer having a functional group for fixing a biologically active substance, the ratio of the ethylenically unsaturated polymerizable monomer having an alkylene glycol residue is not particularly limited in a polymer, and is preferably from 0 to 95% by mole of the total number of repeating units of all monomers in the polymer, more preferably from 30 to 95% by mole thereof, most preferably from 50 to 90% by mole thereof.

In the present invention, examples of the polymerizable functional group introduced to the surface of a core particle include a vinyl group, an allyl group, a methacrylic group, an epoxy group and a styrene group. Among them, a methacrylic group is preferred because of having excellent polymerization capability.

In the present invention, examples of the chain-transfer group introduced to the surface of a core particle include a mercapto group and an amino group. Among them, a mercapto group is preferred because of having excellent reactivity.

The method of introducing the polymerizable functional group or the chain-transfer group to the surface of a particle is not particularly limited, and is preferably a method of forming a covalent bond between a silane coupling agent containing the polymerizable functional group or the chain-transfer group and a functional group on the surface of a core particle.

Examples of the silane coupling agent containing the polymerizable functional group include alkoxysilanes such as (3-methacryloxypropyl)dimethylmethoxysilane, (3-methacryloxypropyl)diethylmethoxysilane, (3-methacryloxypropyl)dimethylethoxysilane, (3-methacryloxypropyl)diethylethoxysilane, (3-methacryloxypropyl)methyldimethoxysilane, (3-methacryloxypropyl)ethyldimethoxysilane, (3-methacryloxypropyl)methyldiethoxysilane, (3-methacryloxypropyl)ethyldiethoxysilane, (3-methacryloxypropyl)trimethoxysilane and (3-methacryloxypropyl)triethoxysilane. From the viewpoint of having excellent reactivity and increasing the amount of a polymer compound to be coated, a trialkoxysilane having a methacrylic group is preferred. In particular, from the viewpoint of reactivity and availability, (3-methacryloxypropyl)trimethoxysilane and (3-methacryloxypropyl)triethoxysilane are preferred. These silane coupling agents are used alone or in combination of two or more thereof.

Examples of the silane coupling agent containing the chain-transfer group include alkoxysilanes such as (3-mercaptopropyl)trimethoxysilane, (3-mercaptopropyl)methyldimethoxysilane, (3-mercaptopropyl)dimethylmethoxysilane, (3-mercaptopropyl)triethoxysilane, (3-mercaptopropyl)methyldiethoxysilane, (3-mercaptopropyl)dimethylethoxysilane, (mercaptomethyl)trimethoxysilane, (mercaptomethyl)methyldimethoxysilane, (mercaptomethyl)dimethylmethoxysilane, (mercaptomethyl)triethoxysilane, (mercaptomethyl)methyldiethoxysilane and (mercaptomethyl)dimethylethoxysilane. From the viewpoint of availability, (3-mercaptopropyl)trimethoxysilane and (3-mercaptopropyl)triethoxysilane are preferred. These mercaptosilane compounds are used alone or in combination of two or more thereof.

No particular limitation is imposed on the method of forming, by means of a silane coupling agent containing the polymerizable functional group or the chain-transfer group, a covalent bond between the polymerizable functional group or the chain-transfer group and a functional group on the surface of a core particle. An example of the method is conducted in such a manner that a silane coupling agent containing the polymerizable functional group or the chain-transfer group is added to an acid aqueous solution at pH 2 to 4 so as to set the concentration thereof to 0.01 to 1.0 mol/L; after stirring the mixture so as to be hydrolyzed, a core particle is added thereto and stirred at 10 to 100° C. for 5 to 180 minutes; the particle is collected by suction filtration and dried; furthermore, the particle is dried by heating at 20 to 100° C. No particular limitation is imposed on the ratio of the core particle to the silane coupling agent containing the polymerizable functional group or the chain-transfer group used herein. Normally, the ratio is such that with respect to the core particle of 1 g, the silane coupling agent containing the polymerizable functional group or the chain-transfer group of 0.1 to 10 mmol is used. The acid aqueous solution is not particularly limited and may be an acetic acid aqueous solution or a hydrochloric acid aqueous solution. In particular, an acetic acid aqueous solution is preferred because it is relatively easy to handle.

After introducing the polymerizable functional group or the chain-transfer group to the surface of the core particle, the particle is mixed with a polymerizable monomer, followed by causing the mixture to develop polymerization reaction. No particular limitation is imposed on the method of conducting these steps. An example of the method is conducted in such a manner that a core particle is added to a solvent in which a polymerizable monomer and a polymerization initiator are dissolved; and the mixture is heated at 0 to 80° C. for 1 to 30 hours while stirring the same. Thereafter, the core particle is collected by filtration under reduced pressure, followed by washing and drying the particle.

No particular limitation is imposed on the ratio between the core particle, polymerizable monomer and polymerization initiator used herein. Normally, the ratio is such that with respect to the core particle of 1 g, the polymerizable monomer of 0.1 to 10 mmol and the polymerization initiator of 0.01 to 10 mmol are used.

The solvent is not limited to any special solvent insofar as each of polymerizable monomers can be dissolved therein. Examples thereof include alcohols such as methanol, ethanol, isopropanol, n-butanol, t-butyl alcohol, n-pentanol; benzene, toluene, tetrahydrofuran, dioxane, dichloromethane, chloroform, cyclohexanone, N,N-dimethylformamide, dimethylsulfoxide, methyl acetate, ethyl acetate, butyl acetate, methyl ethyl ketone, methyl butyl ketone, ethylene glycol monoethyl ether, ethylene glycol monomethyl ether and ethylene glycol monobutyl ether. These solvents are used alone or in combination of two or more thereof.

No particular limitation is imposed on the polymerization initiator. Examples thereof include azo compounds such as 2,2′-azobisisobutylnitrile (hereinafter referred to as “AIBN”) and 1,1′-azobis(cyclohexane-1-carbonitrile), and organic peroxides such as benzoyl peroxide and lauryl peroxide.

In the chemical structure of the polymer formed on the surface of the particle in the present invention, as long as the polymer is a polymer or copolymer derived from at least a polymerizable monomer having a functional group for fixing a biologically active substance, the bonding manner thereof in the case that the polymer is a copolymer may be any manner, such as a random, block or graft manner.

No particular limitation is imposed on the material for the core particle used in the present invention. Whether organic material or inorganic material, any material may be used for the particle. Organic carriers usable herein include a particle composed of a polyacrylamide gel (trade name: Bio-Gel P, manufactured by Bio-Rad Laboratories, Inc.), polystyrene, an ethylene-maleic anhydride copolymer, polymethyl methacrylate or the like, as well as a porous agarose particle (trade name: Sepharose) and a dextran particle (trade name: Sephadex), each of which is used as a carrier in affinity chromatography. On the other hand, preferred inorganic material is an inorganic oxide because a particle thereof has high strength. In particular, silicon oxide is easy to handle and most preferred. The size of the particle is not particularly limited and may be appropriately selected depending on the intended purpose and use. This means that it is possible to produce a particle of any size if the size of the core particle is selected. This point is far more advantageous compared with the method of producing a particle by emulsion polymerization or suspension polymerization in which it is difficult to control the particle size. When any of said particles is actually used, one having a particle diameter from a few nm to 100 μm is preferred, which may vary depending on the intended use.

As aforementioned, the particle of the present invention having a polymer-containing layer formed on the surface thereof is a particle having an excellent capability of fixing a biologically active substance. Moreover, by adding a component containing an alkylene glycol residue to the component of the polymer-containing layer formed on the surface of the particle, it is possible to enhance the property of inhibiting nonspecific adsorption of components other than a target protein. Furthermore, the polymer compound is formed by causing polymerizable functional groups or chain transfer groups forming covalent bonds with the surface of the core particle to develop polymerization reaction, thereby making it possible to cause the polymer compound to be densely grafted onto the surface of the core particle. The grafted particle thus obtained has very low nonspecific adsorption, and the polymer compound does not flow out therefrom in a washing step. Compared with a particle formed by coating a polymer having a functional group in its terminal end onto a core particle and then developing reaction therebetween, the particle of the present invention has an increased amount of the polymer coated on the surface thereof in a chemically/physically stable manner. Therefore, a higher capability of fixing a target biologically active substance is imparted to the particle of the present invention.

Measurement of the amount of the polymer coated on the surface of the particle of the present invention in a chemically/physically stable manner is possible by, for example, electron spectroscopy for chemical analysis (ESCA) or elemental analysis.

For instance, when the core particle comprises silicon dioxide, ESCA measurement realizes comparative determination of the amount of the polymer compound present on the surface of the silicon dioxide particle from the peak intensity ratio (C/Si) between element carbon derived from the polymer compound on the surface of the particle and element silicon derived from the silicon dioxide particle. When the core particle comprises silicon dioxide, the particle of the present invention can achieve that the peak intensity ratio (C/Si) between element carbon derived from the polymer compound on the surface of the particle and element silicon derived from the silicon dioxide particle is 1.0 or more. The upper limit of the peak intensity ratio (C/Si) is not limited, but a rough upper limit of the (C/Si) may be regarded as 5.0 or less. The ESCA measurement referred to herein may be carried out with an X-ray photoelectron spectrometer (such as ESCA5400MC manufactured by ULVAC-PHI, Inc.) under the following condition for example: the analyzed surface area is: 1.0×3.5 mm, the X-ray source is: MgKα X-rays, and the output angle is: 45 deg.

In the case where the core particle comprises inorganic material, the amount of the polymer in the particle of the present invention may be determined as the carbon element content in the particle by elemental analysis. When the core particle comprises inorganic material, the particle of the present invention can achieve that the carbon element content in the particle is 10 to 40% by weight. The elemental analysis referred to herein may be conducted with an elemental analyzer (such as the PerkinElmer 2400 Series II elemental analyzer).

In light of the foregoing, when a biologically active substance is bonded to the particle of the present invention, it is possible to efficiently collect a substance (such as a protein) captured by the biologically active substance. Also, a particle may be suitably used as a particle for analysis, which comprising said particle to which a biologically active substance is fixed through a functional group for fixing a biologically active substance.

Preferably, the method of producing the particle for analysis of the present invention by fixing a biologically active substance to a particle having a polymer-containing layer formed on the surface thereof, comprises the step of bringing a particle having a polymer-containing layer formed on the surface thereof into contact with a solution prepared by dissolving a biologically active substance in a phosphate buffer.

The phosphate buffer is an aqueous solution in which any kind of phosphate is preferably dissolved at a concentration of 0.1 M or more and 5.0 M or less, more preferably 0.6 M or more and 2.4 M or less, most preferably 0.8 M or more and 1.4 M or less. When the concentration is less than the upper limit, there is a risk that a biologically active substance is not sufficiently fixed and no signal is detected. When the concentration is more than the upper limit, there is a risk that a biologically active substance is denatured, so that the biologically active substance does not cause specific reaction and function.

No particular limitation is imposed on the phosphate used in the present invention. Examples thereof include aluminum phosphate, ammonium phosphate, potassium phosphate, sodium phosphate, indium phosphate, samarium phosphate, potassium hydrogen phosphate, dipotassium hydrogen phosphate, calcium hydrogen phosphate, sodium hydrogen phosphate, disodium hydrogen phosphate, ammonium hydrogen phosphate, barium hydrogen phosphate, diammonium phosphate, dipotassium phosphate, 2-aminoethyl dihydrogen phosphate, ammonium dihydrogen phosphate, potassium dihydrogen phosphate, calcium dihydrogen phosphate, sodium dihydrogen phosphate, manganese dihydrogen phosphate, lithium dihydrogen phosphate, dibarium phosphate, hydroxyammonium phosphate, urea phosphate, lithium phosphate, diphenyl phosphate, triethyl phosphate, trioctyl phosphate, triphenyl phosphate, tributyl phosphate, trimethyl phosphate, boron phosphate and magnesium phosphate. Particularly preferred are dipotassium hydrogen phosphate and disodium hydrogen phosphate.

By bringing a particle having a polymer-containing layer formed on the surface thereof into contact with a solution prepared by dissolving a biologically active substance in the phosphate buffer, the biologically active substance can be fixed easily.

Various methods are available for bringing a solution in which a biologically active substance is dissolved into contact with a particle. Examples thereof include a method in which after a particle is charged into a container, a solution is poured thereon in several batches and stirring the same, and a method of bringing a particle into contact with a solution by filling a particle in a column and sending a solution thereto.

The biologically active substance fixed to the particle for analysis of the present invention varies depending on the substance to be captured and collected, so that it is not particularly limited and is preferably a nucleic acid, an aptamer, a protein, an antibody, an antigen, protein A, protein G, a ligand, a peptide, glutathione, a low-molecular compound, biotin, a sugar chain, a lectine, a glycoprotein, a heparin, gelatin, a benzamidine, a lysine, a metal chelate or the like.

As a method of using the particle for analysis of the present invention, there may be mentioned a method of collecting a target biological substance by bringing the particle for analysis into contact with at least one solution selected from a solution in which a target biomolecule is dissolved, blood, blood plasma, blood serum, a cell lysate, a cell culture solution and a tissue lysate.

The method of collecting a target biological substance, which is a substance captured by the biologically active substance fixed to the particle for analysis, is not particularly limited. Examples thereof include a method of releasing a captured substance in such a manner that the particle for analysis is immersed in a sample solution; reaction is developed between a captured substance and a biologically active substance; the captured substance is fixed onto the particle; and the particle on which the captured substance is fixed is immersed in a solution having a controlled salt concentration or pH, a solution containing a surfactant, or a solution containing a chemical specie for developing exchange reaction. The method of determining the amount of a collected substance is not particularly limited, and examples thereof include a method of determining the absorbance or fluorescence of a specific wavelength derived from a captured substance by a spectrophotometrical method and SDS-PAGE.

Using the particle for analysis of the present invention as a column packing material provides a column for refining target molecules from a biological sample. No particular limitation is imposed on the use of the particle for analysis of the present invention as a column, and the particle for analysis may be used as a column for affinity chromatography, reversed phase chromatography, hydrophobic interaction chromatography or the like. The present invention is particularly suitable as a column packing material for affinity chromatography, and the particle may be used as a packing material for open columns, flash columns, spin columns or the like.

Because of the above reasons, the particle for analysis of the present invention may be suitably used as a carrier for refining and collecting a captured substance.

Furthermore, the particle for analysis of the present invention may be suitably used in immune assay for detecting the specificity between an antibody and an antigen, in addition to collection of a captured substance. No particular limitation is imposed on the immune assay referred to herein. Immune assay is a method of analysis using specific reaction between an antibody and an antigen, such as an ELISA method, an EIA method, fluorescence detection, chemiluminescence detection, a radioisotopic method, a condensation method, an immunoprecipitation method and immunochromatography. The particle for analysis of the present invention may be suitably used in any of the above.

Also, the particle for analysis of the present invention may be used as a filler to a micro channel, which may be used in applications such as collection of a substance from an extremely small amount of sample, immune assay and immunochromatography.

### EXAMPLES

### Example I Series

### Production and Evaluation of Particle for Medical Use

(Synthesis of p-Nitrophenyloxycarbonyl-Polyethylene Glycol Methacrylate (MEONP))

Into 20 mL of chloroform, 0.01 mole of polyethylene glycol monomethacrylate (Blenmer PE-200 manufactured by NOF Corp.) was dissolved, and then the solution was cooled to −30° C. While the temperature was maintained at −30° C., into this solution was slowly dropped a homogeneous solution prepared in advance and made of 0.01 mole of p-nitrophenyl chloroformate (manufactured by Aldrich Co.), 0.01 mole of triethylamine (manufactured by Wako Pure Chemical Industries, Ltd.) and 20 mL of chloroform. The reactive components were reacted at −30° C. for 1 hour, and then the solution was further stirred at room temperature for 2 hours. Thereafter, salts were filtrated off from the reaction solution, and the solvent was removed to obtain crude p-nitrophenyloxycarbonyl-polyethylene glycol methacrylate (hereinafter referred to as “MEONP”). The crude product thus obtained was refined with a silica gel column. The resultant monomer was measured by 1H-NMR in deuterated chloroform solvent. As a result, it was confirmed that 4.5 units of ethylene glycol residues were contained.

### Example I-1

Methacryloxypropyltrimethoxysilane (LS3380 manufactured by Shin-Etsu Chemical Co., Ltd.) of 7.45 g was added to 39.3 g of acetic acid aqueous solution at pH 3.0 and stirred at room temperature for 1 hour. A silica bead (5 μm in average particle diameter, 70 Å in pore diameter, SMB70-5 manufactured by Fuji Silysia Chemical Ltd.) of 5 g was added thereto and stirred at 85° C. for 2 hours. Then, the bead was collected from the reaction solution by suction filtration and heated at 100° C. for 1 hour. Thereafter, such a process was repeated two times that the thus-obtained silica bead was dispersed in ethanol, shaken at room temperature for 1 hour, and subjected to centrifugal separation to remove supernatant. Furthermore, such a process was repeated five times that the silica bead was dispersed in ethanol, stirred with a vortex mixer, and subjected to centrifugal separation to remove supernatant. Thereafter, the silica bead was dried.

Polyethylene glycol methyl ether methacrylate having a number-average molecular weight Mn of about 475 (also known as methoxy polyethylene glycol methacrylate, which will be referred to as PEGMA475 hereinafter, manufactured by Aldrich Co.) and MEONP were dissolved in dehydrated ethanol to prepare a monomer mixed solution. The total concentration of the monomers was 0.2 mol/L. About the mole ratio between the individual monomers, the ratio of PEGMA475 to MEONP was 80:20. Furthermore, thereto were added AIBN so as to set the concentration of the component to 0.004 mol/L. The solution was stirred until the solution turned into a homogeneous state. Thereafter, thereto was added 1 g of a silica bead treated with the above-mentioned methacryloxypropyltrimethoxysilane. The reactive components were reacted at 60° C. for 22 hours in the atmosphere of argon gas, and then the silica bead was collected from the reaction solution by suction filtration. Then, such a process was repeated five times that, the silica bead was dispersed in ethanol, and subjected to centrifugal separation to remove supernatant. Thereafter, the bead was collected by suction filtration and dried thoroughly.

### Example I-2

Methacryloxypropyldimethylmethoxysilane (manufactured by Gelest, Inc.) of 13.0 g was added to a solution prepared by mixing 100 g of acetic acid aqueous solution at pH 3.0 with 100 ml of ethanol and stirred at room temperature for 1 hour. A silica bead (5 μm in average particle diameter, 70 Å in pore diameter, SMB70-5 manufactured by Fuji Silysia Chemical Ltd.) of 10 g was added thereto and stirred at 70° C. for 2 hours. Then, the bead was collected from the reaction solution by suction filtration and heated at 100° C. for 1 hour. The subsequent steps of washing, drying, polymerization reaction with MEONP, washing and drying after the polymerization reaction were conducted in the same manner as in Example 1.

### (Evaluation of Amount of Nonspecific Adsorption)

The silica bead obtained in each of Examples I-1 and I-2 of about 37 mg was treated at room temperature for 1 hour in 0.1 mol/L of 2-aminoethanol (solvent: 0.05 mol/L of Tris-HCl buffer at pH 9.5) to inactivate MEONP. After removing supernatant by centrifugal separation, such a process was repeated five times that each resultant was dispersed in a phosphate buffer (PBS), subjected to centrifugal separation to remove supernatant, and dried. The silica bead thus obtained from each resultant of 10 mg was mixed with 5 μg/mL horseradish peroxidase-labeled (hereinafter, horseradish peroxidase will be referred to as HRP) antibody solution (a solution prepared by diluting Polyclonal Rabbit Anti-Mouse Immunogloblins manufactured by DakoCytomation A/S and HRP 260 times with a PBS) of 270 μL. Each mixture was stirred at room temperature for 30 minutes and then subjected to centrifugal separation to remove supernatant. Then, such a process was repeated 15 times that a PBS containing a nonionic surfactant (manufactured by Sigma-Aldrich Co., TritonX100 (T9284-100ML)) at a concentration of 0.05% was added to each resultant to disperse the resultant thoroughly in the PBS; and the resultant was subjected to centrifugal separation to remove supernatant. Thereafter, the silica bead of each resultant was collected with a filter unit (manufactured by Millipore Co., trade mark: Ultrafree-MC). To the collected silica bead, a solution of 3,3′,5,5′-tetramethylbenzidine (hereinafter referred to as TMBZ) (a solution prepared with a peroxidase coloring kit manufactured by Sumitomo Bakelite Co., Ltd.), which was a substrate of HRP, was added and stirred at room temperature for 15 minutes to react TMBZ with the HRP-labeled antibody, which was nonspecifically adsorbed onto the surface of the silica bead. Then, a stop solution (included in a peroxidase coloring kit manufactured by Sumitomo Bakelite Co., Ltd.) was added to stop the reaction. Thereafter, the reaction solution was separated from the silica bead with a filter unit and measured for the absorbance at 450 nm. This absorbance mainly reflects the amount of the HRP-labeled antibody which was nonspecifically adsorbed onto the silica bead.

### (Evaluation of Specific Capture of Protein)

The silica bead obtained in each of Examples I-1 and I-2 of about 37 mg was mixed with 50 μg/mL of an avidin solution (a solution prepared by diluting NeutrAvidin™ Biotin-Binding Protein manufactured by PIERCE in a dipotassium hydrogen phosphate buffer at pH 8.5). After treating each mixture at 37° C. for 4 hours, supernatant was removed by centrifugal separation. Then, such a process was repeated five times that each resultant was dispersed in a phosphate buffer (PBS) and subjected to centrifugal separation to remove supernatant. Additionally, each resultant was treated at room temperature for 1 hour in 0.1 mol/L of 2-aminoethanol (solvent: 0.05 mol/L of Tris-HCl buffer at pH 9.5) to inactivate MEONP. After removing supernatant by centrifugal separation, such a process was repeated five times that each resultant was dispersed in a phosphate buffer (PBS), subjected to centrifugal separation to remove supernatant, and dried. The thus-obtained silica bead of each resultant of 10 mg was mixed with 5 μg/mL biotin-labeled HRP solution (a solution prepared by diluting Biotinylated Peroxidase manufactured by Zymed Laboratories, Inc. 200 times with a PBS) of 270 μL, which solution was capable of specifically binding to avidin, or was mixed with 5 μg/mL HRP-labeled antibody solution of 270 μL. Each mixture was stirred at room temperature for 30 minutes and then subjected to centrifugal separation to remove supernatant. Then, such a process was repeated 15 times that a PBS containing a nonionic surfactant (manufactured by Sigma-Aldrich Co., TritonX100 (T9284-100ML)) at a concentration of 0.05% was added to each resultant to disperse the resultant thoroughly in the PBS; and the resultant was subjected to centrifugal separation to remove supernatant. Thereafter, the silica bead of each resultant was collected with a filter unit (manufactured by Millipore Co., trade mark: Ultrafree-MC). To the collected silica bead, a TMBZ solution (a solution prepared with a peroxidase coloring kit manufactured by Sumitomo Bakelite Co., Ltd.) was added and stirred at room temperature for 15 minutes to react TMBZ with the biotin-labeled HRP, which was specifically captured onto the surface of the silica bead, or with the HRP-labeled antibody, which was nonspecifically adsorbed onto the surface of the silica bead. Then, a stop solution (included in a peroxidase coloring kit manufactured by Sumitomo Bakelite Co., Ltd.) was added to stop the reaction. Thereafter, the reaction solution was separated from the silica bead with a filter unit and measured for the absorbance at 450 nm. When a biotin-labeled HRP solution was used in said process, this absorbance mainly reflects the amount of the biotin-labeled HRP which was specifically captured onto the silica bead. When a HRP-labeled antibody solution was used, this absorbance mainly reflects the amount of the HRP-labeled antibody which was nonspecifically adsorbed onto the silica bead.

### (Analysis of Particle Surface)

ESCA analysis was performed on the silica beads obtained in Examples I-1 and I-2 for the analysis of the surface of the silica bead. ESCA analysis realizes comparative determination of the amount of a polymer compound present on the surface of the silica bead from the peak intensity ratio (C/Si) between element carbon derived from the polymer compound on the surface of the silica bead and the element silicon derived from the silica bead.

### Comparative Example I-1

Using the silica bead (5 μm in average particle diameter, 70 Å in pore diameter, SMB70-5 manufactured by Fuji Silysia Chemical Ltd.) as it is, evaluation of the amount of nonspecific adsorption of proteins and evaluation of specific capturing of a target protein were performed in the same manner as in the above Examples.

### Comparative Example I-2

Polyethylene glycol methyl ether methacrylate having a number-average molecular weight Mn of about 1,100 (also known as methoxy polyethylene glycol methacrylate, which will be referred to as PEGMA1100 hereinafter, manufactured by Aldrich Co.) and MEONP were dissolved in dehydrated ethanol to prepare a monomer mixed solution. The total monomer concentration of the monomers was 0.3 mol/L. About the mole ratio between the individual monomers, the ratio of PEGMA1100 to MEONP was 70:30. Furthermore, thereto were added (3-mercaptopropyl) dimethylethoxysilane (hereinafter referred to as MPDES, manufactured by AZmax Co.) and AIBN so as to set the concentration of each component to 0.003 mol/L. The solution was stirred until the solution turned into a homogeneous state. Thereafter, in the atmosphere of argon gas, the reactive components were reacted at 60° C. for 6 hours, and then the reaction solution was dropped into diethyl ether. The resultant precipitation was then collected. A Silica bead (5 μm in average particle diameter, 70 Å in pore diameter, SMB70-5 manufactured by Fuji Silysia Chemical Ltd.) was added to a 0.3 wt % cyclohexanone solution of the resultant polymer and stirred thoroughly with a vortex mixer. The bead was collected by suction filtration, dried thoroughly, and then subjected to heating treatment at 150° C. for 2 hours.

Surface analysis, evaluation of the amount of nonspecific adsorption, and evaluation of specific capturing of protein were performed on the thus-obtained silica bead in the same manner as in the above Examples.

Table 1 shows the absorbance at 450 nm in the evaluation of the amount of nonspecific adsorption performed on Examples I-1, Example I-2, Comparative Example I-1 and Comparative Example I-2. As is clear from the table, compared to the silica bead of Comparative Example I-1 and the particle of Comparative Example I-2, the amount of nonspecific adsorption of proteins to the particle of Examples I-1 and I-2 of the present invention is significantly lower, so that it is obvious that the nonspecific adsorption of proteins was inhibited in the particle of the present invention.

TABLE 1

| | Example | Example | Comparative | Comparative |
|---|---|---|---|---|
| | I-1 | I-2 | Example I-1 | Example I-2 |
| Adsorbance | 0.04 | 0.08 | 1.56 | 0.87 |
| at 450 nm | | | | |

Table 2 shows the absorbance at 450 nm in the evaluation of specific capturing of protein performed on Examples I-1, Example I-2, Comparative Example I-1 and Comparative Example I-2. As is clear from the table, the particles of the present invention are able to specifically capture a target protein only, while inhibiting nonspecific adsorption of proteins.

TABLE 2

Adsorbance at 450 nm

| | Biotin-Labeled HRP | HRP-Labeled Antibody |
|---|---|---|
| Example I-1 | 1.66 | 0.08 |
| Example I-2 | 1.24 | 0.08 |
| Comparative | 1.42 | 1.46 |
| Example I-1 | | |
| Comparative | 0.80 | 0.84 |
| Example I-2 | | |

Table 3 shows the result of ESCA surface analysis of Example I-1, Example I-2, and Comparative Example I-2. The particles of the present invention showed higher C/Si values than that of the silica bead in Comparative Example I-2; thus, it is obvious that the particles of the present invention were coated with a larger amount of polymer than the particle comprising a silica bead coated with a preliminarily polymerized polymer.

TABLE 3

| | | | Comparative |
|---|---|---|---|
| | Example I-1 | Example I-2 | Example I-2 |
| C/Si | 2.7 | 1.2 | 0.3 |

### Example II Series

### Production and Evaluation of Particle for Analysis

### Example II-1

A solution was prepared in which a primary antibody, anti-mouse IgG2a, was contained at a concentration of 12 μg/mL in a 0.5 M dipotassium hydrogen phosphate (manufactured by Wako Pure Chemical Industries, Ltd.: 164-04295) aqueous solution. Into 500 μL of this solution, the silica bead obtained in Example I-1 of about 10 mg was added and stirred at 37° C. for 4 hours to fix the primary antibody. After removing supernatant by centrifugal separation, such a process was repeated five times that the resultant was dispersed in a phosphate buffer (PBS) and subjected to centrifugal separation to remove supernatant. Then, in 0.1 mol/L of 2-aminoethanol (solvent: 0.05 mol/L of Tris-HCl buffer at pH 9.5), the resultant was treated at room temperature for 1 hour to inactivate MEONP. After removing supernatant by centrifugal separation, such a process was repeated five times that the resultant was dispersed in a phosphate buffer (PBS) and subjected to centrifugal separation to remove supernatant. The resultant was dried to obtain a particle for analysis.

### Example II-2

The same process as in Example II-1 was carried out except that a 1.2 M dipotassium hydrogen phosphate (manufactured by Wako Pure Chemical Industries, Ltd.: 164-04295) aqueous solution was used.

### Example II-3

The same process as in Example II-1 was carried out except that a 2.4 M dipotassium hydrogen phosphate (manufactured by: Wako Pure Chemical Industries, Ltd.: 164-04295) aqueous solution was used.

### Comparative Example II-1

Said silica bead (5 μm in average particle diameter, 70 Å in pore diameter, SMB70-5 manufactured by Fuji Silysia Chemical Ltd.) was used as it is.

### Comparative Example II-2

In 0.1 mol/L of 2-aminoethanol (solvent: 0.05 mol/L of Tris-HCl buffer at pH 9.5), the silica bead obtained in Example I-1 of about 37 mg was treated at room temperature for 1 hour to inactivate MEONP. After removing supernatant by centrifugal separation, such a process was repeated five times that the resultant was dispersed in a phosphate buffer (PBS) and subjected to centrifugal separation to remove supernatant. Thereafter, the resultant was dried.

### Comparative Example II-3

The same process as in Example II-1 was carried out except that the silica bead obtained in Comparative Example I-2 was used.

### Evaluation 1

### (Antigen-Antibody Reaction 1)

An FBS (fetal bovine serum) solution was prepared by diluting to 10% with a PBS buffer (a buffer wherein 9.6 g of tissue-culturing Dulbecco's PBS(−) (:manufactured by Nissui Pharmaceutical Co., Ltd.) was dissolved in 1 L of pure water). To this solution was added the mouse IgG2a as the antigen to produce a solution in which the concentration thereof was 20 nmol/L. This solution was diluted with the FBS (fetal bovine serum) solution diluted to 10% with a PBS buffer (a buffer wherein 9.6 g of tissue-culturing Dulbecco's PBS(−) (:manufactured by Nissui Pharmaceutical Co., Ltd.) was dissolved in 1 L of pure water) one time, 2 times, 3 times and 4 times, so as to yield diluted solutions. Each of these diluted solutions of 1 ml and the 10% FBS solution of 1 ml, in which no mouse IgG2a was contained as the antigen, were each brought into contact with 1 mg of the particle for analysis obtained in each of Examples II-1 to II-3 and Comparative Examples II-1 to II-3 at room temperature for 2 hours, thereby developing antigen-antibody reaction. After the antigen-antibody reaction, supernatant was removed by centrifugal separation. Thereafter, such a process was repeated five times that each resultant was dispersed in a phosphate buffer (PBS) to which a 0.05% by weight of nonionic surfactant Tween 20 (manufactured by Roche Diagnostics K.K.) was added, and subjected to centrifugal separation to remove supernatant.

### (Antigen-Antibody Reaction 2)

A HRP-labeled anti-mouse IgG2a as a second antibody was added to a PBS buffer (a buffer wherein 9.6 g of tissue-culturing Dulbecco's PBS(−) (:manufactured by Nissui Pharmaceutical Co., Ltd.) was dissolved in 1 L of pure water), thereby preparing a solution in which the concentration thereof was 20 nmol/L. Antigen-antibody reaction was developed at room temperature for 2 hours between 1 ml of this solution and 1 mg of the bead obtained from each resultant. After the antigen-antibody reaction, such a process was repeated five times that each resultant was dispersed in a phosphate buffer (PBS) to which a 0.05% by weight of nonionic surfactant Tween 20 (manufactured by Roche Diagnostics K.K.) was added, and subjected to centrifugal separation to remove supernatant.

### (Coloring)

Finally, coloring reaction was carried out with a TMBZ coloring kit (manufactured by: Sumitomo Bakelite Co., Ltd.), which is a HRP coloring reagent. A coloring solution was prepared by mixing 100 parts by volume of a coloring agent and 1 part by volume of a substrate solution. Each of obtained bead of 1 mg was added to the coloring solution of 100 μl, and placed in the dark for 15 minutes. Thereafter, a stop solution of 100 μl was added to stop the coloring reaction. The colored solutions thus obtained were measured for the absorbance at 450 nm with a plate reader manufactured by TECAN.

The results of signal intensity are shown in Table 4.

In Examples II-1 to II-3, as a result of fixing the antibody with the antigen of certain concentrations, signal values corresponding to the amounts of the antigen were observed. In Comparative Example II-1, nonspecific antigen adsorption was not inhibited since no surface treatment was conducted. In Comparative Example II-2, no signal was obtained since the primary antibody was not fixed. In Comparative Example II-3, nonspecific antigen adsorption was not inhibited since the preliminarily-polymerized polymer was not sufficiently coated onto the silica bead.

TABLE 4
Signal Intensity

| | Dilution Ratio | | | | |
|---|---|---|---|---|---|
| | | | | | No |
| | 1 | 2 | 3 | 4 | Antigen |
| Example II-1 | 2.03 | 1.31 | 0.92 | 0.78 | 0.05 |
| Example II-2 | 2.63 | 1.50 | 1.19 | 0.95 | 0.06 |
| Example II-3 | 2.55 | 1.49 | 1.04 | 0.81 | 0.05 |
| Comparative | 2.13 | 1.87 | 1.67 | 1.54 | 1.44 |
| Example II-1 | | | | | |
| Comparative | 0.06 | 0.05 | 0.05 | 0.04 | 0.05 |
| Example II-2 | | | | | |
| Comparative | 2.23 | 2.02 | 2.47 | 2.13 | 2.41 |
| Example II-3 | | | | | |

### Evaluation 2

### (Antigen-Antibody Reaction 1)

A human serum solution was prepared by diluting to 10% with a PBS buffer (a buffer wherein 9.6 g of tissue-culturing Dulbecco's PBS(−) (:manufactured by Nissui Pharmaceutical Co., Ltd.) was dissolved in 1 L of pure water). To this solution was added the mouse IgG2a as the antigen to produce a solution in which the concentration thereof was 20 nmol/L. This solution was diluted with a human serum solution diluted into 10% with a PBS buffer (a buffer wherein 9.6 g of tissue-culturing Dulbecco's PBS(−) (:manufactured by Nissui Pharmaceutical Co., Ltd.) was dissolved in 1 L of pure water) 4 times so as to yield a diluted solution. This diluted solution of 1 ml was brought into contact with the particle for analysis obtained in each of Examples II-1 to II-3 and Comparative Examples II-1 to II-3 at room temperature for 2 hours, thereby developing antigen-antibody reaction. After the antigen-antibody reaction, supernatant was removed by centrifugal separation. Thereafter, such a process was repeated five times that each resultant was dispersed in a phosphate buffer (PBS) to which a 0.05% by weight of nonionic surfactant Tween 20 (manufactured by Roche Diagnostics K.K.) was added, and subjected to centrifugal separation to remove supernatant.

### (SDS-PAGE)

The bead thus obtained from each resultant was dispersed in 30 μl of a glycine-hydrochloric acid solution (pH 2.5) and subjected to centrifugal separation to collect supernatant. A 1:1 mixture of each supernatant thus obtained and a laemmli buffer (0.25 M Tris at pH 6.8, 6% SDS (sodium dodecyl sulfate), 40% glycerin, 0.04% bromophenol blue aqueous solution) was heated at 90° C. for 5 minutes. After cooling, each of the resultant solutions was subjected to electrophoresis at 200V for 45 minutes. Gels which exhibited electrophoretic activity were silver stained (Silver Staining MS Kit 299-58901 manufactured by Wako Pure Chemical Industries, Ltd.) to obtain bands for analysis.

In Examples II-1 to II-3, rat albumin-derived bands were observed around the molecular weight of 69 kDa. In Comparative Example II-1, many human serum-derived bands were observed since no surface treatment was conducted. No band was observed in Comparative Example II-2. In Comparative Example II-3, many human serum-derived bands were observed since the preliminarily-polymerized polymer was not sufficiently coated onto the silica bead.

### Example II-4

A solution was prepared in which an oligo-DNA with a chain length of 24 by having an amino group at the 5′ terminal (TAGAAGCATTTGCGGTGGACGATG (sequence number 1) manufactured by Sigma-Genosys) was contained at a concentration of 1 μg/μl in a 0.5 M dipotassium hydrogen phosphate (manufactured by Wako Pure Chemical Industries, Ltd.: 164-04295) aqueous solution. Into 500 μL of this solution, the silica bead obtained in Example I-1 of about 10 mg was added and stirred at 37° C. for 4 hours to fix the DNA. After removing supernatant by centrifugal separation, such a process was repeated five times that the resultant was dispersed in a phosphate buffer (PBS) and subjected to centrifugal separation to remove supernatant. Then, in 0.1 mol/L of 2-aminoethanol (solvent: 0.05 mol/L of Tris-HCl buffer at pH 9.5), the resultant was treated at room temperature for 1 hour to inactivate MEONP. After removing supernatant by centrifugal separation, such a process was repeated five times that the resultant was dispersed in a phosphate buffer (PBS) and subjected to centrifugal separation to remove supernatant. The resultant was then dried.

### Example II-5

The same process as in Example II-4 was carried out except that a 1.2 M dipotassium hydrogen phosphate (manufactured by Wako Pure Chemical Industries, Ltd.: 164-04295) aqueous solution was used.

### Example II-6

The same process as in Example II-4 was carried out except that a 4.5 M dipotassium hydrogen phosphate (manufactured by Wako Pure Chemical Industries, Ltd.: 164-04295) aqueous solution was used.

### Comparative Example II-4

The same process as in Example II-4 was carried out except that the silica bead obtained in Comparative Example I-2 was used.

### Evaluation 3

DNA solution 2; an oligo-DNA with a chain length of 24 by having a biotin label at the 5′ terminal (CATCGTCCACCGCAAATGCTTCTA (sequence number 2) manufactured by Sigma-Genosys) was dissolved at the concentration 0.002 μg/μl in 3×SSC—0.2% SDS (the SSC referred to herein means a SSC Buffer 20× Concentrate (S6639-1L manufactured by Sigma-Genosys) diluted 20 times and the composition thereof comprises 0.015 mol/l sodium citrate and 0.15 mol/l sodium chloride; and the 3×SSC referred to herein means a SSC condensed three times).

Then, this solution was brought into contact with 1 mg of the particle for analysis obtained in each of Examples II-4 to II-6 and Comparative Examples II-1, II-2 and II-4, and stirred at 65° C. for 3 hours for hybridization of the fixed oligo-DNA and biotin-labeled oligo-DNA. Then, after removing supernatant by centrifugal separation, such a process was repeated five times that each resultant was dispersed in 2×SSC-0.5% SDS and subjected to centrifugal separation to remove supernatant. Further, each of bead thus obtained was dispersed in a 0.1 μg/ml streptavidin solution and stirred at room temperature for 30 minutes. After removing supernatant by centrifugal separation, such a process was repeated five times that each resultant was dispersed in a phosphate buffer (PBS) to which a 0.05% by weight of nonionic surfactant Tween 20 (manufactured by Roche Diagnostics K.K.) was added, and subjected to centrifugal separation to remove supernatant.

### (Coloring)

Finally, coloring reaction was carried out with a TMBZ coloring kit (manufactured by: Sumitomo Bakelite Co., Ltd.), which is a HRP coloring reagent. A coloring solution was prepared by mixing 100 parts by volume of a coloring agent and 1 part by volume of a substrate solution. Each of obtained bead of 1 mg was added to the coloring solution of 100 μl, and placed in the dark for 15 minutes. Thereafter, a stop solution of 100 μl was added to stop the coloring reaction. The colored solutions thus obtained were measured for the absorbance at 450 nm with a plate reader manufactured by TECAN. The results of signal intensity are shown in Table 5.

TABLE 5

| | Signal Intensity |
|---|---|
| Example II-4 | 2.57 |
| Example II-5 | 2.69 |
| Example II-6 | 2.85 |
| Comparative Example II-1 | 0.07 |
| Comparative Example II-2 | 0.06 |
| Comparative Example II-4 | 1.47 |

Examples show higher signal amounts than those of Comparative Examples.

### Example II-7

A solution was prepared in which a peptide with a chain length of 18 mer (CERIKIKALIPKNAGVSD) (manufactured by Immuno-Biological Laboratories Co., Ltd.) was contained at a concentration of 10 μg/μl in a 0.5 M dipotassium hydrogen phosphate (manufactured by Wako Pure Chemical Industries, Ltd.: 164-04295) aqueous solution. Into 500 μL of this solution, the silica bead obtained in Example I-1 of about 10 mg was added and stirred at 37° C. for 4 hours to fix the peptide. After removing supernatant by centrifugal separation, such a process was repeated five times that the resultant was dispersed in a phosphate buffer (PBS) and subjected to centrifugal separation to remove supernatant. Then, in 0.1 mol/L of 2-aminoethanol (solvent: 0.05 mol/L of Tris-HCl buffer at pH 9.5), the resultant was treated at room temperature for 1 hour to inactivate MEONP. After removing supernatant by centrifugal separation, such a process was repeated five times that the resultant was dispersed in a phosphate buffer (PBS) and subjected to centrifugal separation to remove supernatant. The resultant was then dried.

### Example II-8

The same process as in Example II-7 was carried out except that a 1.2 M dipotassium hydrogen phosphate (manufactured by Wako Pure Chemical Industries, Ltd.: 164-04295) aqueous solution was used.

### Example II-9

The same process as in Example II-7 was carried out except that a 4.5 M dipotassium hydrogen phosphate (manufactured by Wako Pure Chemical Industries, Ltd.: 164-04295) aqueous solution was used.

### Comparative Example II-5

The same process as in Example II-7 was carried out except that the silica bead obtained in Comparative Example 1-2 was used.

### Evaluation 4

The peptide with a chain length of 18 mer (CERIKIKALIPKNAGVSD) was administered to a rabbit so that the rabbit gains immunity against this peptide, thereby producing antibody against the peptide. The thus-obtained antiserum was evaluated.

A rabbit antiserum solution was prepared by diluting to 10% with a PBS buffer (a buffer wherein 9.6 g of tissue-culturing Dulbecco's PBS(−) (:manufactured by Nissui Pharmaceutical Co., Ltd.) was dissolved in 1 L of pure water). This diluted solution of 1 ml was brought into contact with the particle for analysis obtained in each of Examples II-7 to II-9 and Comparative Examples II-1, II-2 and II-5 at room temperature for 2 hours, thereby developing antigen-antibody reaction. After the antigen-antibody reaction, supernatant was removed by centrifugal separation. Thereafter, such a process was repeated five times that each resultant was dispersed in a phosphate buffer (PBS) to which a 0.05% by weight of nonionic surfactant Tween 20 (manufactured by Roche Diagnostics K.K.) was added, and subjected to centrifugal separation to remove supernatant.

### (SDS-PAGE)

The bead thus obtained from each resultant was dispersed in 30 μl of a glycine-hydrochloric acid solution (pH 2.5) and subjected to centrifugal separation to collect supernatant. A 1:1 mixture of each supernatant thus obtained and a laemmli buffer (0.25 M Tris at pH 6.8, 6% SDS, 40% glycerin, 0.04% bromophenol blue aqueous solution) was heated at 90° C. for five minutes. After cooling, each of the resultant solutions was subjected to electrophoresis at 200V for 45 minutes. Gels which exhibited electrophoretic activity were silver stained (Silver Staining MS Kit 299-58901 manufactured by Wako Pure Chemical Industries, Ltd.) to obtain bands for analysis.

In Examples II-7 to II-9, bands were observed around the molecular weight of 50 kDa, which bands were derived from the H chain of the antibody.

Additionally, bands were observed around the molecular weight of 25 kDa, which bands were derived from the L chain of the antibody. In Comparative Example II-1, many rabbit serum-derived bands were observed, as well as the bands derived from the H and L chains of the antibody. No band was observed in Comparative Example II-2. In Comparative Example II-5, many rabbit serum-derived bands were observed since the preliminarily-polymerized polymer compound was not sufficiently coated onto the silica bead.

1. A particle for medical use which has on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop polymerization reaction.

2. The particle for medical use according to claim 1, wherein the polymerizable component contains an ethylenically unsaturated polymerizable monomer having an alkylene glycol residue.

3. The particle for medical use according to claim 1 or 2, wherein the functional group of the polymerizable monomer having a functional group for fixing a biologically active substance is at least one functional group selected from an aldehyde group, an active ester group, an epoxy group, a vinylsulfone group and biotin.

4. The particle for medical use according to claim 1, wherein the polymerizable monomer having a functional group for fixing a biologically active substance is a monomer having an active ester group and represented by the following formula [1]: 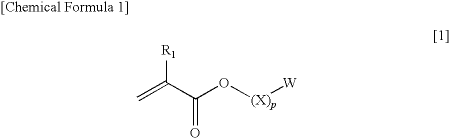
wherein R₁ represents a hydrogen atom or a methyl group; X represents an alkylene group or an alkylene glycol residue having 1 to 10 carbon atoms; W represents an active ester group; p represents an integer from 1 to 100; and provided that when p is an integer of 2 or more and 100 or less, the repeated Xs may be the same or different from each other.

5. The particle for medical use according to claim 4, wherein the active ester group is a p-nitrophenyl ester or N-hydroxysuccinimide ester.

6. The particle for medical use according to any of claim 2, wherein the ethylenically unsaturated polymerizable monomer having an alkylene glycol residue contains a monomer represented by the following formula [2]: 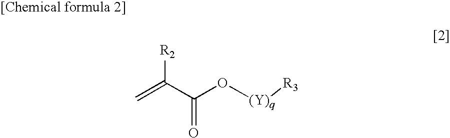
wherein R₂ represents a hydrogen atom or a methyl group; R₃ represents a hydrogen atom or an alkyl group having 1 to 20 carbon atoms; Y represents an alkylene glycol residue having 1 to 10 carbon atoms; q represents an integer from 1 to 100; and provided that when q is an integer of 2 or more and 100 or less, the repeated Ys may be the same or different from each other.

7. The particle for medical use according to claim 2, wherein the ethylenically unsaturated polymerizable monomer having an alkylene glycol residue contains methoxy polyethylene glycol (meth)acrylate and/or ethoxy polyethylene glycol (meth)acrylate.

8. The particle for medical use according to claim 7, wherein an average repeating number of the ethylene glycol residue of the methoxy polyethylene glycol (meth)acrylate and/or ethoxy polyethylene glycol (meth)acrylate is from 3 to 100.

9. The particle for medical use according to claim 1, wherein the polymerizable functional group is one or more kinds selected from the group consisting of a methacrylic group, an acrylic group and a vinyl group.

10. The particle for medical use according to claim 1, wherein the chain-transfer group is a mercapto group.

11. The particle for medical use according to any of claim 1, wherein the core particle comprises an inorganic material.

12. The particle for medical use according to claim 11, wherein the inorganic material comprises an inorganic oxide.

13. The particle for medical use according to claim 12, wherein the inorganic oxide is a silicon oxide.

14. The particle for medical use according to claim 1, wherein the polymerizable functional group or the chain-transfer group is introduced to the surface of the core particle by forming a covalent bond between a silane coupling agent containing the polymerizable functional group or the chain-transfer group and a functional group on the surface of the core particle.

15. The particle for medical use according to claim 14, wherein the silane coupling agent containing the polymerizable functional group or the chain-transfer group is an alkoxysilane containing the polymerizable functional group or the chain-transfer group.

16. A method of producing the particle for medical use defined by claim 1, which method comprises the steps of hydrolyzing an alkoxysilane containing the polymerizable functional group or the chain-transfer group in an acid aqueous solution, heating the core particle while stirring the same in the acid aqueous solution of the alkoxysilane containing the polymerizable functional group or the chain-transfer group, and further heating the particle after drying the same.

17. The method of producing the particle for medical use according to claim 16, which method further comprising the steps of mixing in a solvent the core particle to which the polymerizable functional group or the chain-transfer group is introduced with a polymerizable monomer to develop polymerization reaction, and drying the particle.

18. The method of producing the particle for medical use according to claim 17, wherein the polymerization reaction is a radical polymerization reaction.

19. A particle for analysis of the interaction of biomolecules, which particle having on the surface thereof a polymer-containing layer formed by introducing a polymerizable functional group or a chain-transfer group to the surface of a core particle, mixing the resultant particle with a polymerizable component containing a polymerizable monomer having a functional group for fixing a biologically active substance, and causing the mixture to develop polymerization reaction, wherein the biologically active substance is fixed through the functional group for fixing a biologically active substance in the polymer-containing layer.

20. The particle for analysis according to claim 19, wherein the polymerizable component contains an ethylenically unsaturated polymerizable monomer having an alkylene glycol residue.

21. The particle for analysis according to claim 19, wherein the functional group for fixing a biologically active substance is at least one functional group selected from the group consisting of an aldehyde group, an active ester group, an epoxy group, a vinylsulfone group and biotin.

22. The particle for analysis according to claim 19, wherein the biologically active substance is at least one selected from the group consisting of a nucleic acid, an aptamer, a protein, an antibody, an antigen, protein A, protein G, a ligand, a peptide, glutathione, a low-molecular compound, biotin, a sugar chain, a lectine and a glycoprotein.

23. A method of producing the particle for analysis defined by claim 19, which method comprises the step of bringing the particle having a polymer-containing layer formed on the surface thereof into contact with a solution prepared by dissolving a biologically active substance in a phosphate buffer.

24. The method of producing the particle for analysis according to claim 23, wherein the concentration of the phosphate in the phosphate buffer is 0.1 M or more and 5 M or less.

25. The method of producing the particle for analysis according to claim 23, wherein the phosphate is potassium dihydrogen phosphate, sodium dihydrogen phosphate, dipotassium hydrogen phosphate or disodium hydrogen phosphate.

26. A method of using the particle for analysis defined by claim 19, which method comprises collecting a target biological substance by bringing the particle for analysis defined by claim 19 into contact with at least one solution selected from a solution in which a target biomolecule is dissolved, blood, blood plasma, blood serum, a cell lysate, a cell culture solution and a tissue lysate.
